(19) United States
(12) Patent Application Publication
(10) Pub. No.: US 2023/0355481 A1
(43) Pub. Date: Nov. 9, 2023

(54) PEG Feeding Tube Support and Comfort System

(71) Applicant: Joanna Lee Rodriguez, Destin, FL (US)

(72) Inventor: Joanna Lee Rodriguez, Destin, FL (US)

(21) Appl. No.: 18/143,959

(22) Filed: May 5, 2023

Related U.S. Application Data
(60) Provisional application No. 63/339,217, filed on May 6, 2022.

(51) Int. Cl. A61J 15/00

(52) U.S. Cl. CPC A61J 15/0061; A61J 15/0015; A61M 2025/0206

(57) ABSTRACT

A PEG tube support and comfort system for providing support for a patient's PEG feeding tube and comfort for the patient. The PEG tube support and comfort system including a unique pillow-like, padded support device that is worn by the patient at or near the incision site. The support device including a flat portion that rests against the waist of the patient and a crescent-shaped, padded, pillow-like portion that extends outwardly from the flat portion and which provides a support or resting place for the PEG tube. The flat portion including a pouch or pocket which provides protected space for the patient to place and store the extra PEG tubing. The PEG tube and comfort system also including a strap that is wrapped around the waist area of the patient and which holds the support device in place against the waist of the patient.

### CROSS-REFERENCE TO RELATED APPLICATION

This application claims the benefit of U.S. Provisional Application No. 63/339,217 entitled “Provides Exterior Comfort and Support for a PEG Feeding Tube,” filed on May 5, 2022, which is hereby incorporated by reference in its entirety.

### TECHNOLOGICAL FIELD

This application relates generally to the field of percutaneous endoscopic gastrostomy (“PEG”), a procedure in which a flexible feeding tube is placed through the abdominal wall of a patient and into the stomach. The PEG procedure allows nutrition, fluids and/or medications to be put directly into the stomach, bypassing the mouth and esophagus. Specifically, this patent application relates to a device that can hold and provides support for the PEG tube on the outside of the patient's body and thus increases comfort for the patient.

### BACKGROUND OF THE INVENTION

During a percutaneous endoscopic gastrostomy (PEG), a doctor typically uses a lighted flexible tube called an endoscope to guide the creation of a small opening through the skin of the upper abdomen and directly into the stomach. This procedure allows the doctor to place and secure a feeding tube into the stomach. Specialized liquid nutrition, as well as fluids, are given through the PEG tube. Patients who have difficulty swallowing, problems with their appetite, or an inability to take adequate nutrition through the mouth can benefit from this procedure. In this way, a PEG is extremely beneficial to a patient needing an alternative way to receive nutrition, fluids, and/or medications.

Although there obviously is a benefit to this procedure, it can also lead to significant discomfort and even pain for the patient. There is nothing inherent to the PEG tube to hold or support the tube itself where it exits the body. Thus, discomfort and pain can result from the PEG tube pulling at the surgical site due to the tube protruding from the body, causing this pain and discomfort. Existing devices do not provide any support to the tube itself where it exits the body. The existing devices allow the tube to move, kink, be pulled, or catch on clothing or other items, all of which therefore puts pressure on and causes pain and discomfort at the incision site.

### BRIEF SUMMARY OF THE INVENTION

The claimed invention solves the problem described above associated with PEG tubes and existing devices used in connection with PEG tubes by providing a unique PEG tube support and comfort system. The system comprises a unique pillow-like, padded support device that is worn by the patient at or near the incision site. The support device comprises a flat portion that rests against the waist of the patient and a crescent-shaped, padded, pillow-like portion that extends outwardly from the flat portion and which provides a support or resting place for the PEG tube. The flat portion is created to form a pouch or pocket which provides protected space for the patient to place and store the extra tubing. The system also comprises an adjustable strap or belt that is wrapped around the waist area of the patient and which holds the support device in place. The design of the unique pillow-like device provides a small crescent-shaped pillow that functions as a support for the PEG tube. By supporting the PEG tube, the device prevents the tube from moving, kinking, pulling, or catching on clothing or other items. By preventing the various, potential movements of the PEG tube, the system prevents the pain and discomfort that can result from the movement of the PEG tube. In addition to providing support for the PEG tube, the pouch or pocket formed by the flat portion which provides an enclosed, protected space to hold the extra tubing all while laying flush against the waist of the patient.

### BRIEF DESCRIPTION OF THE DRAWINGS

These and other features, aspects, and advantages of the present invention will become better understood with regard to the following description, appended claims, and accompanying drawings where:

FIG. 1 is a drawing of the support device of the present invention.

FIG. 2 is a drawing of the support device of the present invention showing the inside of the pouch of the support device.

FIG. 3 is a drawing of the support device of the present invention showing the inside of the pouch of the support device.

FIG. 4 is a drawing of the strap portion of the PEG tube support and comfort system

FIG. 5 is a drawing of the strap portion of the PEG tube support and comfort system

FIG. 6 is a drawing of the PEG tube support and comfort system

FIG. 7 is a drawing of the PEG tube support and comfort system

FIG. 8 is a drawing of the PEG tube support and comfort system as worn by a patient

### DETAILED DESCRIPTION OF THE INVENTION

With reference now to the drawings, a PEG tube support and comfort system is herein described.

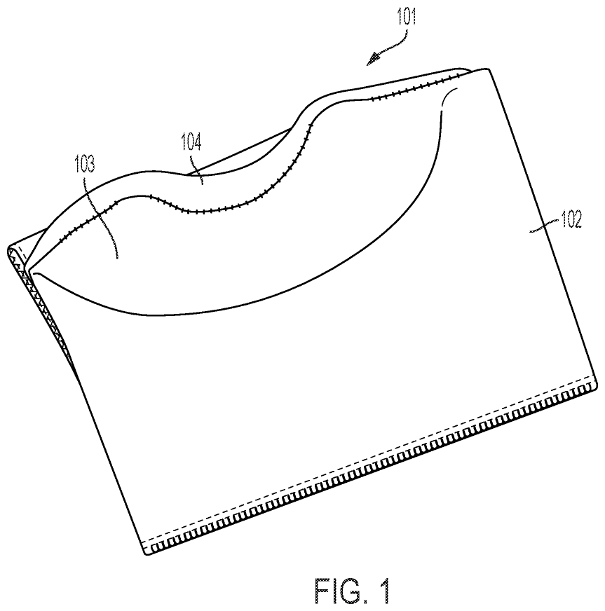

As shown in FIGS. 1-3, the PEG tube support and comfort system of the present invention comprises a unique pillow-like, padded support device 101. The support device 101 is preferably made from a soft fabric that is comfortable to be worn against the skin of a patient and preferably washable. The support device 101 comprises a flat portion 102. When the support device 101 is worn by the patient, the flat portion 102 lies against the skin of the patient, preferably near the abdominal area of the patient where the PEG incision site is located. Along the upper portion of the support device 101 at the top of the flat portion 102 is a pillow-like, padded portion 103. Because of the padding associated with the portion 103, that portion extends out from the flat portion 102. When the support device 101 is worn by the patient, the padded portion 103 also lies against the skin of the patient, just below the PEG incision site and preferably centered along the PEG incision site. The padded portion 103 is uniquely shaped to provide a support area 104. When the support device is worn by the patient, the support area 104 is positioned just below the PEG incision site, and the support area 104 is where the PEG tube is placed. By resting on the support area 104, the PEG tube is supported and held in place, thereby preventing any pain or discomfort to the patient from the PEG tube moving, pulling, kinking, or catching on clothing or other objects. Preferably, the shape of the padded portion 103 is a crescent or similar shape where the support area 104 is recessed from the other areas of the top of the padded portion 103 thereby creating the support area 104 for the PEG tube. The recessed portion allows the PEG tube to be held or supported in place and to prevent its movement, pulling, kinking, or catching.

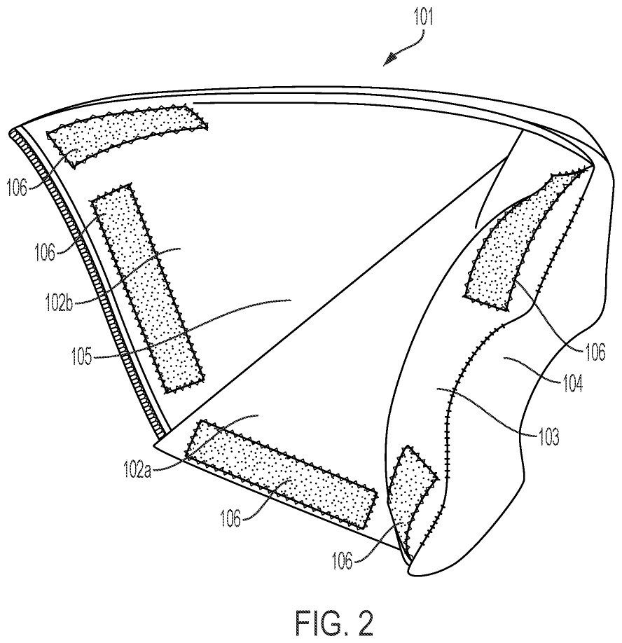

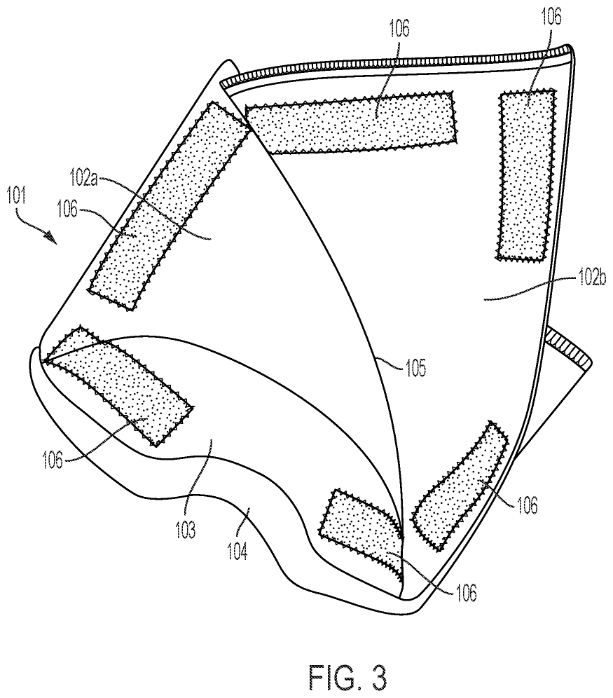

As shown in FIGS. 2-3, the flat portion 102 of the support device 101 may be designed so as to form a pouch (or pocket) 105. To create such a pouch 105, the flat portion 102 would include two portions—flat portion 102a which is attached to or integral with the padded portion 103 and a flat portion 102b connected to flat portion 102a on the bottom of the support device 101 and on one side of the support device 101 but detached on one side of the support device 101 thereby creating the pouch 105. The pouch 105 can be used by the patient to hold or store excess PEG tubing, thereby preventing the excess tubing from moving, pulling, kinking, or catching, thereby creating a possible danger for the patient. The flat portion 102a and the flat portion 102b preferably include attachment means for temporarily closing the pouch so that the excess PEG tube is held into place. In FIGS. 2-3, such attachment means is shown as Velcro strips 106, but the attachment means can be anything that would hold the flat portions 102a and 102b together to close the pouch 105 to hold in place the excess PEG tubing. The other markings on FIGS. 1-3 represent stitching that may exist from sewing together various pieces of fabric to form the support device 101 and its various sections, including the flat portion(s) and the padded portion.

It should be noted that the support device 101 can be made of any size to maximize the comfort for the patient. Smaller patients might prefer a smaller support device while larger patients might be able to accommodate a larger support device. The relative size of the support device should not impact the overall functionality of the device as long as the support area 104 is sufficiently defined and large enough to accommodate the PEG tube.

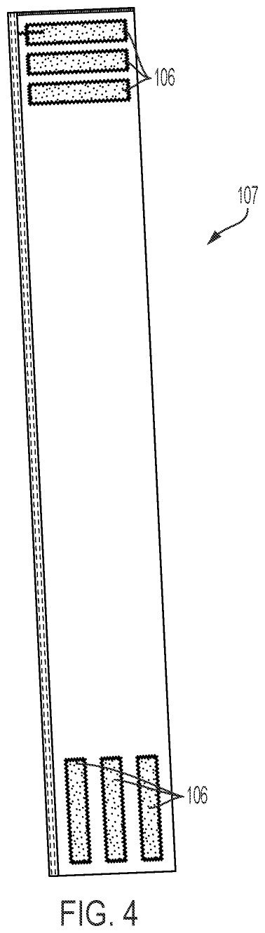

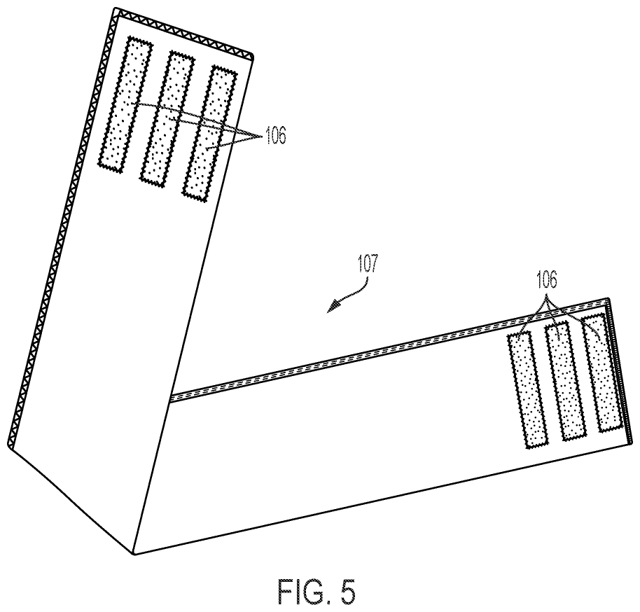

As shown in FIGS. 4-5, the PEG tube support and comfort system of the present invention preferably comprises an adjustable strap 107. The adjustable strap 107 is preferably made from a soft fabric that is comfortable to be held against the skin of a patient. The adjustable strap 107 is preferably a rectangular shape and is sufficiently long to wrap around the waist of a patient. As shown in FIGS. 4-5, the adjustable strap 107 comprises attachment means to hold the strap 107 together and in place when wrapped around the waist of the patient. In FIGS. 4-5, such attachment means is shown as Velcro strips 106, but other methods of attaching one end of the adjustable strap to the other end as it is wrapped around the patient's waist may be used as well. The attachment means, such as the Velcro strips 106, are placed in appropriate positions to maximize the security of the adjustable strap 107 and thus the support device 101.

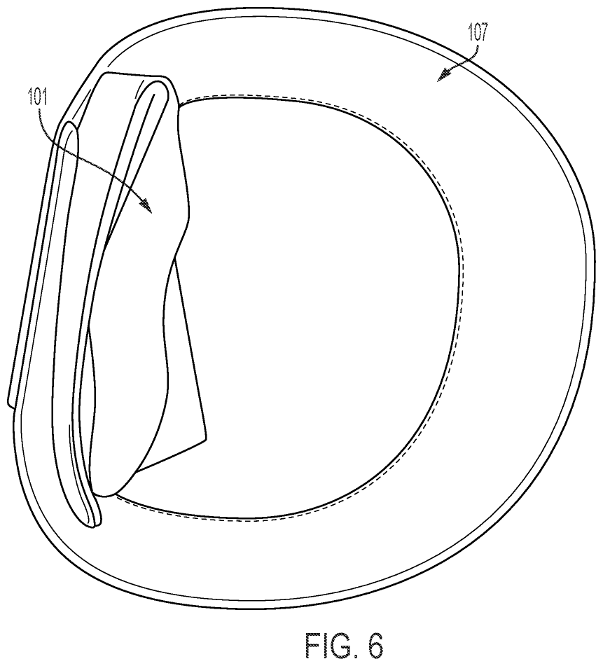

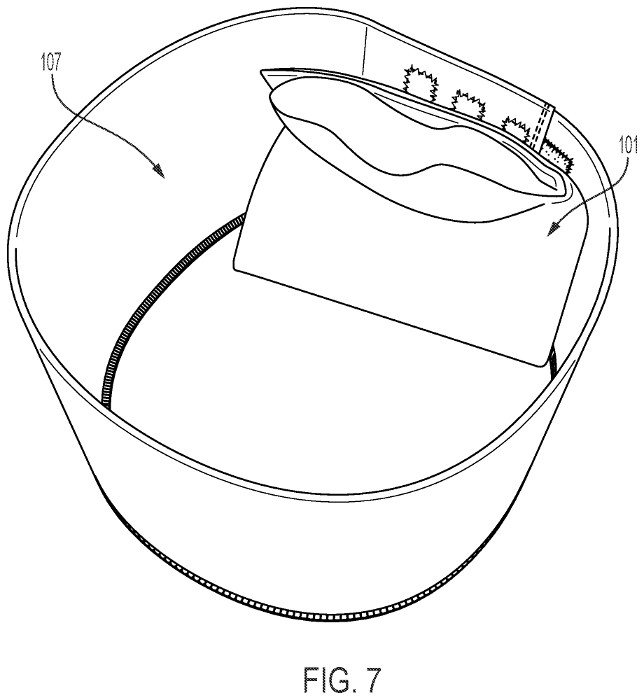

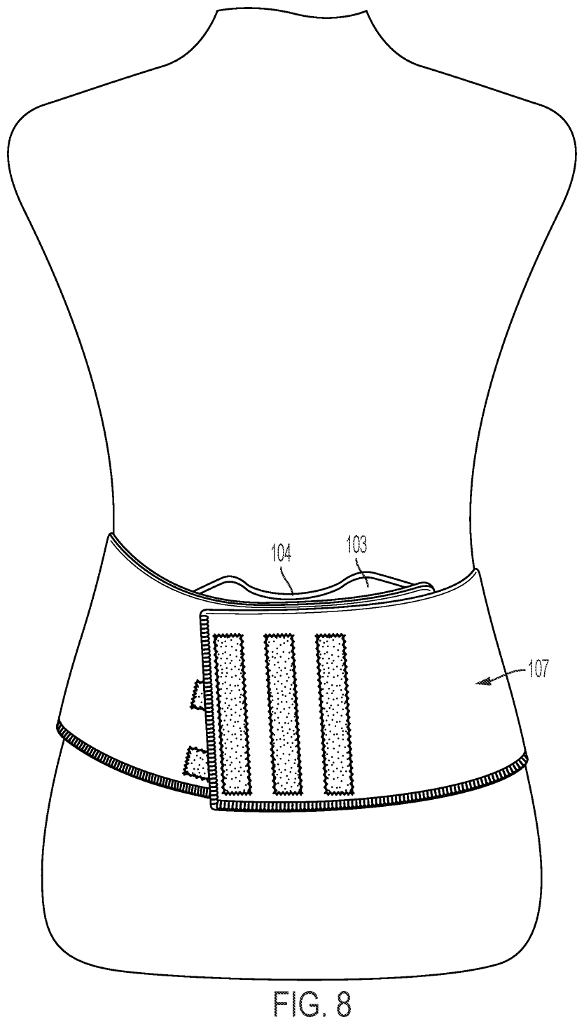

As shown in FIG. 6-7, the adjustable strap 107 is closed and formed into a circular ring using the attachment means and the support device 101 is placed on the inside of the adjustable strap 107. In this way, the support device 101 is held in place by the adjustable strap 107 against the patient's abdominal area near the incision site and where the PEG tube is inserted into the patient's abdomen into the patient's stomach. FIG. 8 shows the support device 101 held in place against the patient's abdominal area by the adjustable strap 107.

In a further embodiment of the claimed invention, the flat portion 102b and the adjustable strap 107 each include a small opening, or slit, through which a PEG tube could be inserted to allow the PEG feeding tube support and comfort system to be worn and used by the patient while the PEG feeding tube is actively used for delivering medication, nourishment, or similar. When being worn by the patient, the patient would align the small opening or slit in the adjustable strap 107 near the small opening or slit in the flat portion 102b.

It should be appreciated that the novel PEG tube support and comfort system provides padded support for the PEG tube where the tube exits/enters the body and also minimizes excess bulk under the patient's clothing to secure the PEG tubing. The preferable soft fabric used for the support device 101 and the adjustable strap 107 is suitable for long-term contact with the skin of the patient and also machine washable. Because of the nature of the preferred fabric, it also is able to absorb any liquids that might be present as a result of the PEG. A further benefit of the claimed invention is that the adjustable strap 107 secures the support device 101 against the patient's abdomen for discreet appearance, allowing the patient not only to be free from pain or discomfort from the PEG tube, but also less anxious about having the PEG and PEG tubing.

The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components.

Although the present invention has been described in considerable detail with possible reference to certain preferred versions thereof, other versions are possible. Therefore, the spirit and scope of the appended claims should not be limited to the description of the preferred versions contained herein. All features disclosed in this specification may be replaced by alternative features serving the same, equivalent, or similar purpose, unless expressly stated otherwise. Thus, unless expressly stated otherwise, each feature disclosed is one example only of a generic series of equivalent or similar features. Further, it is not necessary for all embodiments of the invention to have all the advantages of the invention or fulfill all the purposes of the invention.

In the present description, the claims below, and in the accompanying drawings, reference is made to particular features of the invention. It is to be understood that the disclosure of the invention in this specification includes all possible combinations of such particular features. For example, where a particular feature is disclosed in the context of a claim, that feature can also be employed, to the extent possible, in aspects and embodiments of the invention, and in the invention generally.

Also, although the description above contains many specificities, these should not be construed as limiting the scope of the embodiments but as merely providing illustrations of some of several embodiments. Thus, the scope of the embodiments should be determined by the appended claims and their legal equivalents, rather than by the examples given.

What is claimed is:

1. A PEG feeding tube comfort and support system, comprising:
a support device comprising a flat portion and a padded portion, where the padded portion extends outward from the flat portion and is shaped to form a support area for supporting a PEG feeding tube; and
an adjustable strap capable of forming a belt for holding the support device in place.

2. The PEG feeding tube comfort and support system of claim 1, wherein the flat portion of the support device comprises a first layer and a second layer where the first layer and second layer are connected to each other so as to form a pouch for storing excess PEG feeding tube.

3. The PEG feeding tube comfort and support system of claim 2, wherein the first layer and second layer of the flat portion further comprise temporary attachment means for opening and closing the pouch.

4. The PEG feeding tube comfort and support system of claim 3, wherein the attachment means of the first layer and second layer of the flat portion comprises Velcro strips.

5. The PEG feeding tube comfort and support system of claim 1, wherein the support device and the adjustable strap are made of a soft fabric.

6. The PEG feeding tube comfort and support system of claim 1, wherein the adjustable strap comprises temporary attachment means for opening and closing the belt formed by the adjustable strap.

7. The PEG feeding tube comfort and support system of claim 6, wherein the temporary attachment means of the adjustable strap comprises Velcro strips.

8. The PEG feeding tube comfort and support system of claim 1, where the padded portion is crescent shaped and where the top of the padded portion is recessed to form the support area.

9. The PEG feeding tube comfort and support system of claim 1, wherein the flat portion of the support device and the adjustable strap each further comprise an opening through which a PEG feeding tube could be inserted.
